(19) United States
(12) Patent Application Publication
(10) Pub. No.: US 2025/0197461 A1
(43) Pub. Date: Jun. 19, 2025

(54) Insect-derived short peptide for improving abscisic acid (ABA) content in cruciferous plant

(71) Applicant: Gannan Normal University, Ganzhou City (CN)

(72) Inventors: Wei CHEN, Ganzhou City (CN); Yuan LIAO, Ganzhou City (CN); Yu XIONG, Ganzhou City (CN); Qian GUO, Ganzhou City (CN); Mengjie WU, Ganzhou City (CN); Hongjing ZHONG, Ganzhou City (CN); Xin REN, Ganzhou City (CN); Ruijun XU, Ganzhou City (CN); Xin ZHONG, Ganzhou City (CN); Zhanjun LU, Ganzhou City (CN)

(21) Appl. No.: 18/602,739

(22) Filed: Mar. 12, 2024

(30) Foreign Application Priority Data
Mar. 13, 2023 (CN) 202310232365.6

(51) Int. Cl. C07K 14/435; A01H 3/00; C12N 9/02; C12N 9/16; C12N 15/10

(52) U.S. Cl. CPC C07K 14/43563; A01H 3/00; C12N 9/0077; C12N 9/16; C12N 15/1055; C12Y 301/05

(57) ABSTRACT

In the present disclosure, a yeast interactive plasmid of a detoxifying enzyme glucosinolate sulfatase (GSS1) is constructed based on a yeast two-hybrid system and then screened with an Arabidopsis thaliana-yeast library, and a plant protein zeaxanthin epoxidase aba1 that interacts with the GSS1 is obtained for the first time. A zeaxanthin epoxidase gene, also known as an aba-1 gene, is an abscisic acid (ABA) synthase that can catalyze oxidative conversion of the zeaxanthin into all-trans violaxanthin, thereby generating carotenoid precursors required in an ABA biosynthetic pathway. Furthermore, a core peptide segment that interacts with the aba1 is obtained by continuously reducing the length of a GSS1 sequence and conducting one-to-one yeast two-hybrid verification with the aba1 sequence.

### CROSS REFERENCE TO RELATED APPLICATION

This patent application claims the benefit and priority of Chinese Patent Application No. 202310232365.6 filed with the China National Intellectual Property Administration on Mar. 13, 2023, the disclosure of which is incorporated by reference herein in its entirety as part of the present application.

### REFERENCE TO SEQUENCE LISTING

A computer readable XML file entitled “Sequence Listing”, that was created on Apr. 29, 2024, with a file size of 22,469 bytes, contains the sequence listing for this application, has been filed with this application, and is hereby incorporated by reference in its entirety.

### TECHNICAL FIELD

The present disclosure belongs to the technical field of use of insect-derived short peptides, and specifically relates to an insect-derived short peptide for improving abscisic acid (ABA) content in a cruciferous plant.

### BACKGROUND

Abscisic acid (ABA), one of the six major plant hormones, serves as a phytohormone that inhibits growth and is named due to inducing leaves to fall off. The ABA may be widely distributed in higher plants. In addition to inducing leaves to fall off, ABA has other functions, such as inducing buds enter a dormant state and potatoes to form tubers. The ABA also has an inhibitory effect on cell elongation. Meanwhile, under abiotic stress conditions, plants can improve stress resistance by accumulating ABA, making the ABA also known as a “stress hormone”. Therefore, the ABA shows a greater potential to regulate plant growth in the face of stress. Currently, commercial products, such as commercial sodium tungstate, focus on inhibitors to reduce ABA. The sodium tungstate can inhibit the activity of ABA aldehyde oxidase in the ABA synthesis process, such that ABA aldehyde cannot be converted into the ABA.

When insects feed on plants, they interact with proteins encoded by important genes in the plants through oral secretions. Once plants sense “molecular cues” of insects, they stimulate hormone signaling pathways to build defenses. The composition of insect oral secretory proteins is diverse and complex, which play an important role in regulating plant hormone-mediated defense. Insect oral secretory proteins can be mainly divided into two categories, namely elicitors and effectors. In general, the elicitors stimulate plant defense responses, while the effectors inhibit plant defense responses. There are no reports on looking for peptide that regulate plant ABA hormones based on oral secretion proteins of insects. The oral secretory proteins of Plutella xylostella have not yet been fully characterized. However, it is found in previous studies that a detoxifying enzyme glucosinolate sulfatase (GSS1) in vivo can be released into wounds when the Plutella xylostella feed on plants. However, whether this protein is an elicitor or an effector has not been fully explored.

### SUMMARY

The purpose of the present disclosure is to provide an insect-derived short peptide for improving an ABA content in a cruciferous plant, so as to solve the technical problem that sodium tungstate, a compound medicament, is used for regulating plant ABA hormones in the prior art, which has certain adverse effects on the environment and human body.

In order to solve the above technical problems, a short peptide with a length of 28 amino acids derived from Plutella xylostella is identified for the first time in the present disclosure, which can improve the ABA hormone content. Moreover, the increase of the ABA hormone content synergistically affects jasmonic acid (JA) hormone, indicating that this peptide segment shows a certain potential to enhance plant stress resistance in the future. It is specifically as follows:

In the present disclosure, an insect-derived short peptide is provided for improving an ABA content in a cruciferous plant, with an amino acid composition of

| (SEQ ID NO: 1) | |
|---|---|
| | PDYKQKLRDSKAWESLETIGIPLDADVM. |

Further, a method for preparing the insect-derived short peptide for improving the ABA content in the cruciferous plant includes the following steps:
S1, constructing a yeast bait plasmid of a detoxifying enzyme glucosinolate sulfatase (GSS1) based on a yeast two-hybrid system;
S2, screening the yeast bait plasmid of the GSS1 with an Arabidopsis thaliana-yeast library to obtain a plant protein zeaxanthin epoxidase aba1 that interacts with the GSS1; and
S3, screening a shortest interactive short peptide between the GSS1 and the aba1 based on a yeast one-to-one hybrid system.

Compared with the prior art, beneficial effects of the technical scheme of the present disclosure are as follows:

In the present disclosure, a yeast interactive plasmid of a detoxifying enzyme glucosinolate sulfatase (GSS1) is constructed based on a yeast two-hybrid system and then screened with an Arabidopsis thaliana-yeast library, and a plant protein zeaxanthin epoxidase aba1 that interacts with the GSS1 is obtained for the first time. A zeaxanthin epoxidase gene, also known as an aba-1 gene, is an abscisic acid (ABA) synthase that can catalyze oxidative conversion of the zeaxanthin into all-trans violaxanthin, thereby generating carotenoid precursors required in an ABA biosynthetic pathway. Further, by continuously reducing the length of the GSS1 sequence and conducting one-to-one yeast two-hybrid verification with the aba1 sequence, a core peptide segment with a length of 28 amino acids (PDYKQKLRDSKAWESLETIGIPLDADVM, SEQ ID NO: 1) interacting with the aba1 is obtained, named GSS1-PI short peptide. Through the Arabidopsis thaliana smear experiment with artificially synthetic short peptides corresponding to a length of 28 amino acids, using detection method, it is revealed that the core sequence of 28-amino acid of GSS1 in Plutella xylostella can interact with the ABA hormone synthase aba1, and significantly improve the ABA content and a resulting hormone JA content in Arabidopsis thaliana. The increase of the content of plant endogenous hormones further up-regulated the content of flavonoids against insects, which in turn led to an increase in the mortality of Plutella xylostella larval.

### BRIEF DESCRIPTION OF THE DRAWINGS

FIG. 1 shows the results of GSS1 self-activation detection.

FIG. 2 shows the transformation efficiency statistics of the GSS1 screening library.

FIG. 3 shows the plant genes based on potential interactions between yeast two-hybrid GSS1 and Arabidopsis thaliana.

FIG. 4 shows the interaction between positive clone retransformation verification and GSS1.

FIG. 5 shows the screening of the shortest short peptide of the GSS1 and aba1.

FIGS. 6A-6H show the hormones detection results of Arabidopsis thaliana after treating with GSS1-PI and GFP control short peptide.

FIG. 7 shows the biological observation results of Plutella xylostella after feeding on Arabidopsis thaliana treated with GSS1-PI and GFP control short peptide.

### DETAILED DESCRIPTION OF THE EMBODIMENTS

The present disclosure will be further described below with reference to the accompanying drawings and specific examples, but the described examples are merely some rather than all of the examples of the present disclosure. All other embodiments obtained by those skilled in the art based on the embodiments of the present disclosure without creative efforts shall fall within the protection scope of the present disclosure.

In the present disclosure, an insect-derived short peptide is provided for improving an ABA content in a cruciferous plant, with an amino acid composition of PDYKQKLRDSKAWESLETIGIPLDADVM (SEQ ID NO: 1). The method for preparing the insect-derived short peptide includes the following steps:
S1, constructing a yeast bait plasmid of a detoxifying enzyme glucosinolate sulfatase (GSS1) based on a yeast two-hybrid system;
S2, screening the yeast bait plasmid of the GSS1 with an Arabidopsis thaliana-yeast library to obtain a plant protein zeaxanthin epoxidase aba1 that interacts with the GSS1; and
S3, screening a shortest interactive short peptide between the GSS1 and the aba1 based on a yeast one-to-one hybrid system.

### Example 1 Method for Preparing an Insect-Derived Short Peptide for Improving an ABA Content in a Cruciferous Plant

### S1, Construction of a GSS1 Bait Plasmid and its Self-Activation Detection

### (1) Construction of a GSS1 Bait Plasmid

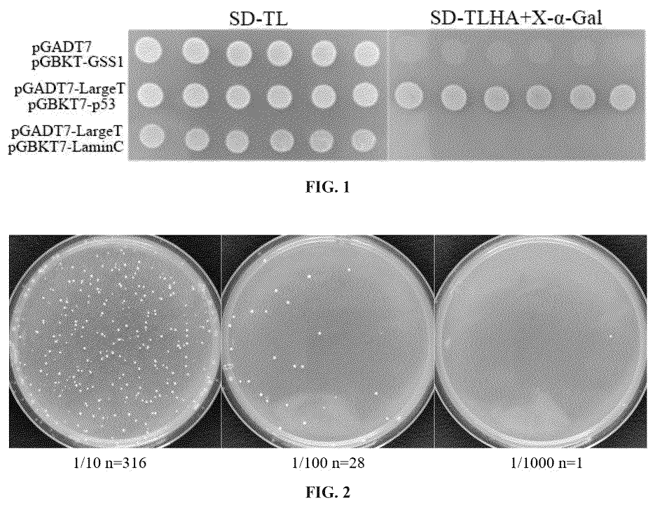

Referring to FIG. 1 to FIG. 4, one-step directional cloning primers (Table 1) were designed to amplify a GSS1 gene, double enzyme digestion was conducted to linearize a pGBKT7 vector, the insert fragment and the linearized vector were recombined using a Hieff Clone® Plus One Step Cloning Kit, and a GSS1-pGBKT7 recombinant bait plasmid was obtained through blue-white plaque screening and sequencing. The vector double enzyme digestion system (Table 1) and homologous recombination reaction system (Table 2) were as follows:

TABLE 1
Vector double enzyme digestion system

| Reagent | Volume | |
|---|---|---|
| Plasmid | ≤1 | μg |
| Endonuclease 1 | 1 | μL |
| Endonuclease 2 | 1 | μL |
| 10 × buffer | 2 | μL |

| | |
|---|---|
| ddH₂O | Up to 20 μL |

TABLE 2
Homologous recombination reaction system

| Reagent | Volume | |
|---|---|---|
| 2 × Hieff Clone ® Enzyme Premix | 10 | μL |
| Inserted segment | X | μL |
| Linearized vector | Y | μL |

| | |
|---|---|
| ddH₂O | Up to 20 μL |

(2) Transformation of the Bait Plasmid into Y2HGold Yeast Cells

The constructed plasmid was transformed and spread on SD/−Trp solid medium, including commercial positive and negative control plasmids. Various plasmid transformations were shown in Table 3.

TABLE 3
Yeast transformation reaction

| | AD | BD | Plate | |
|---|---|---|---|---|
| Reaction | plasmid | plasmid | type | Note |
| 1 | pGADT7- | pGBKT7-p53 | SD-TL | Positive |
| | largeT | | | control |
| 2 | pGADT7- | pGBKT7- | SD-TL | Negative |
| | largeT | laminC | | control |
| 3 | pGADT7 | pGBKT7-GSS1 | SD-TL | Self-activation |
| | | | | detection |

### (3) Yeast Transformation Method

1) a single colony was picked from the YPDA plate and inoculated into 4 mL of YPDA liquid medium, and then cultured with shaking at 30° C. and 225 rpm overnight for 18 h to 20 h until the OD₆₀₀ was about 4;
2) the colony was transferred into a new YPDA liquid medium at a culture volume of 50 mL to achieve initial OD₆₀₀=0.2, and cultured at 30° C. and 225 rpm for 4 h to 5 h with shaking until OD₆₀₀=0.6;
3) yeast cells cultured for 4 h to 5 h in step 2) were collected by centrifugation at room temperature, 4,000 rpm for 5 min, and the collected yeast cells were resuspended in 20 mL of sterile water, mixed well, the obtained mixture was centrifuged at room temperature, 4,000 rpm for 5 min to obtain yeast cells-2, and a resulting supernatant was discarded;
4) the yeast cells-2 were resuspended in 5 mL of 0.1M LiAc, mixed well, and the obtained mixture was centrifuged at room temperature, 4,000 rpm for 5 min to obtain yeast cells-3, and a resulting supernatant was discarded;
5) the yeast cells-3 were resuspended in 500 μL of 0.1M LiAc, mixed well, and the obtained mixture containing yeast cells-3 was packaged into 1.5 mL centrifuge tubes, 50 μL for each transformation for later use;
6) 240 μL of 50% PEG3350, 36 μL of 1 M LiAc, 5 μL of ssDNA, and 5 μL each of plasmid DNA (i.e. AD plasmids and BD plasmids in Table 3) were added to each of the 1.5 mL centrifuge tubes, and shaken vigorously for 1 min until completely mixed;
7) a resulting mixture was incubated in a 30° C. water bath for 30 min, heat-shocked in a 42° C. water bath for 25 min, resuscitated in a 30° C. water bath for 30 min. The recovered mixture was centrifuged at room temperature, 4,000 rpm for 5 min to collect yeast cells, and a resulting supernatant was discarded;
8) the yeast cells were suspended in 200 μL of sterile water for each transformation, mixed as gently as possible, the resulting suspension was spread on a corresponding defective screening plate, and incubated at 30° C. for 4 d.

### (4) Self-Activation Detection of GSS1

The pGBKT7-GSS1 and pGBKT7 empty vectors were streaked on SD/−Trp, SD/−Trp/−His, and SD/−Trp/−Ade plates separately, and cultured at 30° C. for 2 d to 4 d to observe whether the yeast colonies grew. If the colony was not grow (or 3-AT could effectively inhibit growth), it indicated that there was no self-activation activity, and the next experiment could be conducted. After detection according to the self-activation detection method, the results were shown in FIG. 1. In FIG. 1, the yeast containing the bait protein and the yeast containing the pGBKT7 empty plasmid only grew on the 2-deficient medium (SD/−Trp/−Leu), but did not grow on the 4-deficient medium (SD/−Trp/−Leu/−His/−Ade). This indicated that the pGBKT7-GSS1 had no self-activation.

S2, Screening of GSS1 with Arabidopsis thaliana-Yeast Library

### (1) Library Screening

A Y2HGold yeast transformant containing the correct pGBKT7-GSS1 bait plasmid was used as a recipient strain to prepare competent cells, and a library plasmid extracted from the yeast library was transferred into the competent cells, and coated on the SD-Trp-Leu-His+5 mM3AT plate. The specific transformation method of library DNA was as follows:
1) Monoclonal strains were selected from the SD-Trp plate and inoculated into 50 mL of SD-Trp liquid medium, and cultured at 30° C. and 225 rpm with shaking for 18 h.
2) The strains were transferred into a YPDA liquid medium (500 mL) to achieve initial OD₆₀₀=0.2, and cultured at 30° C. and 225 rpm for 4 h to 5 h with shaking until OD₆₀₀=0.6.
3) Yeast cells were collected by centrifugation at room temperature, 4,000 rpm for 5 min, the collected yeast cells were resuspended in 30 mL of sterile water, mixed well, the obtained mixture was centrifuged at room temperature, 4,000 rpm for 5 min to obtain yeast cells-A, and a resulting supernatant was discarded.
4) The yeast cells-A were resuspended in 20 mL of 0.1M LiAc, mixed well, and the obtained mixture was centrifuged at room temperature, 4,000 rpm for 5 min to obtain yeast cells-B, and a resulting supernatant was discarded.
5) The yeast cells-B were resuspended in 10 mL of 0.1M LiAc, mixed well, and the obtained mixture was centrifuged at room temperature, 4,000 rpm for 5 min to obtain yeast cells-C, and a resulting supernatant was discarded.
6) 9.6 mL of 50% PEG3350, 1.44 mL of 1M LiAc, 300 μL of ssDNA, and 25 μg of library DNA plasmid were added to the centrifuge tube in sequence, and shaken vigorously for about 1 min until completely mixed.
7) A resulting mixture was incubated in a 30° C. water bath for 30 min, heat-shocked in a 42° C. water bath for 25 min, and resuscitated in a 30° C. water bath for 1 h.
8) The resuscitated yeast cells were collected by centrifugation at room temperature, 4,000 rpm for 5 min, a resulting supernatant was discarded, and the collected resuscitated yeast cells were resuspended in 8 mL of sterile water, and mixed as gently as possible. 20 μL of the culture was collected and diluted through gradients, and the culture after gradient dilution was coated with 3 SD-TL plates to test the library transformation efficiency. The remaining cultures were coated with SD-TLH+5 mM 3AT, 200 μL per plate, 40 plates in total.
9) The coated cultures were cultured at a constant temperature of 30° C. for 3 d to 4 d to observe the transformation results.

Referring to FIG. 2, the bait and library were cultured in 2×YPDA liquid medium at a constant temperature for 20 h (yeast sexual reproduction mating method), and 10 μL of the mixed yeast solution was taken and continued to be cultured for 0 h to 4 h under an ordinary optical microscope before subsequent experiments were conducted. According to the statistical results, the conversion efficiency was calculated to be 3.7×10³/μg according to the formula, which met the requirements of the screening library and could be used for subsequent experiments.

The single colony grown in the above step was selected onto the QDO solid medium containing x-α-gal, and continue to culture at 30° C. for 3 d to 6 d; this step was repeated once. Single colonies that grew on QDO and turned blue were selected for yeast liquid PCR. After agarose gel electrophoresis, the corresponding PCR products with inserted fragments were selected for sequencing. The sequencing results were aligned by using NCBI Blast, and gene functions were annotated.

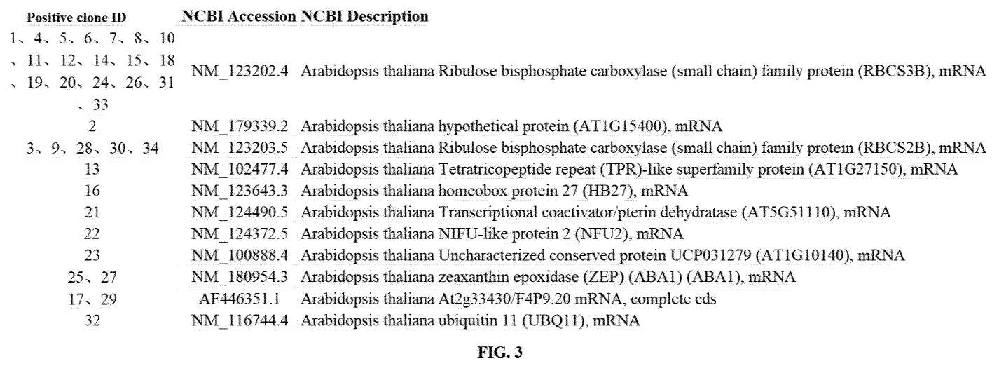

Referring to FIG. 3, the blue single colony growing on the QDO (containing x-α-gal) solid medium for the second culture was selected for yeast liquid PCR; the corresponding PCR product with an insert fragment of more than 750 bp was selected for sequencing, and all the sequencing results were aligned by using Blast. The alignment results showed that most of the prey proteins obtained by screening were undefined proteins. In addition, there were potential interacting proteins obtained by screening, among which ABA1 was closely related to the abscisic acid hormone and became a focus of research in the present disclosure.

### (2) Yeast Retransformation Verification

A yeast transformant containing the correct pGBKT7-GSS1 bait plasmid was used as a recipient strain to prepare competent cells, and 11 positive clone plasmids were transformed into the competent cells (the transformation method was the same as above) and spread on SD-TL plates. The transformants grown on the above-mentioned SD-TL-deficient plates were diluted with sterile water and inoculated onto SD-TL+X-α-Gal and SD-TLHA-deficient plates, and cultured at a constant temperature of 30° C. for 3 d to 4 d. The interaction between two proteins was screened to verify.

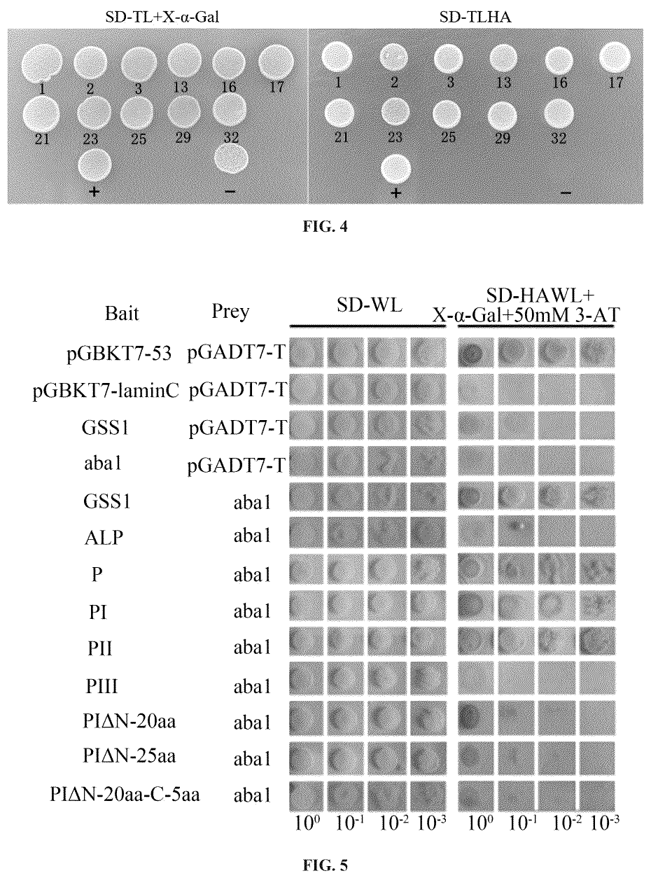

Referring to FIG. 4, the results showed that the retransformation verification of all the 11 positive clones could activate the HIS3, ADE2, and MEL1 reporter genes, indicating that the 11 positive clones could interact with the oral secretory protein GSS1 of Plutella xylostella.

### S3, Screening of the Shortest Short Peptide of the GSS1 and Aba1

### (1) Prediction of the Conserved Site of the Interaction Segment Between GSS1 and Aba1

This experiment was conducted to explore which region of the GSS1 amino acid sequence was required for interaction with aba1. The structural and functional domains of the GSS1 protein were predicted using the online website NCBI, and a total of 8 variants were constructed based on the prediction and screening results, including: GSS1-ALP, GSS1-P, GSS1-PI, GSS1-PII, GSS1-PIII, GSS1-PIΔN-20aa, GSS1-PIΔN-25aa, and GSS1-PIΔN-20aa-C-5aa.

(2) Two-Hybrid Verification of GSS1 Variants with Different Deletion Lengths with Aba1
(i) Construction of GSS1 Variant Vectors with Different Deletion Lengths

GSS1 gene variants and aba1 gene fragments were amplified using one-step directional cloning primers (Table 4), and subjected to homologous recombination with linearized vectors pGBKT7 and pGADT7, respectively. The vector construction method was the same as above.

TABLE 4
Yeast two-hybrid verification of one-step
directional cloning primers

| Primer | Sequence |
|---|---|
| aba1-AD-F | ccatggaggccagtgaattcATGGGTTCAACTCCGTTTTG |
| | CTA (SEQ ID NO: 2) |
| aba1-AD-R | agctcgagctcgatggatccAGCTGTCTGAAGTAATTTAT |
| | CG (SEQ ID NO: 3) |
| gss1-BD-F | tggccatggaggccgaattcATGGCCACCAAGCCCCAC |
| | (SEQ ID NO: 4) |
| gss1-BD-R | cgctgcaggtcgacggatccTTCGGAGTCGGAGTAgGGTT |
| | (SEQ ID NO: 5) |
| gss1-ALP- | tggccatggaggccgaattcatgGCCACCAAGCCCCAC |
| BD-F | (SEQ ID NO: 6) |
| gss1-ALP- | cgctgcaggtcgacggatccGGTTCCCTTGACGAGCTTGT |
| BD-R | (SEQ ID NO: 7) |
| gss1-P- | tggccatggaggccgaattcatgATCGACGAGTCTCTCAG |
| BD-F | CA (SEQ ID NO: 8) |
| gss1-PI- | cgctgcaggtcgacggatccCATGACGTCAGCGTCCAGA |
| BD-R | (SEQ ID NO: 9) |
| gss1-PII- | tggccatggaggccgaattcatgGCTGACCGCGATGAG |
| BD-F | (SEQ ID NO: 10) |
| gss1-PII- | cgctgcaggtcgacggatccAGCCAGCTGAGGAAGCTC |
| BD-R | (SEQ ID NO: 11) |
| gss1-PIII- | tggccatggaggccgaattcatgCAGATCCTTCTGTACCG |
| BD-F | TC (SEQ ID NO: 12) |
| gss1-PI- | tggccatggaggccgaattcATGGAAGACCTCCGTGGCAT |
| 10aa-BD-F | (SEQ ID NO: 13) |
| gss1-PI- | tggccatggaggccgaattcATGCCAGACTACAAGCAGAA |
| B20aa-D-F | GC (SEQ ID NO: 14) |
| gss1-PII- | cgctgcaggtcgacggatccACGAAGCTCACAAGGGTCCT |
| 10aa-BD-R | (SEQ ID NO: 15) |
| gss1-PI- | tggccatggaggccgaattcATGAAGCTGCGCGACAGCAA |
| 25aa-BD-F | (SEQ ID NO: 16) |
| gss1-PI- | cgctgcaggtcgacggatccCAGAGGGATGCCGATGGT |
| 20aa-BD5aa- | (SEQ ID NO: 17) |
| R | |
| pGBKT7-F | TAATACGACTCACTATAGGGC (SEQ ID NO: 18) |
| pGBKT7-R | TTTTCGTTTTAAAACCTAAGAGTC (SEQ ID NO: 19) |

### (ii) Two-Hybrid Verification to Obtain the Shortest Interaction Site of GSS1

The two recombinant plasmid vectors were transferred into Y2HGold yeast competent cells using the co-transfection method, and the Y2HGold yeast competent cells transferred into vectors were screened and verified in auxotrophic medium TDO and QDO (containing x-α-gal). Specific steps were as follows:

The transformation of competent yeast cells was conducted according to the instruction manual (Coolaber) with minor modifications: an appropriate amount of single-stranded carrier DNA was denatured in a 99° C. water bath for 5 min, placed on ice for 2 min, and repeated once; a pre-cooled plasmid (about 3 μg), 10 μL Carrier DNA, and 500 μL PEG/LiAc were added to 100 μL Y2HGold yeast competent cells in sequence; a resulting mixture was treated in a 30° C. water bath for 30 min (turning over 6 to 8 times every 15 min), and then in a 45° C. water bath for 15 min (turning over 6 to 8 times every 7.5 min); after the water bath treatment was completed, centrifugation was conducted at 5,000 g for 40 s and a supernatant was removed; 400 μL of sterile water was added to resuspend the yeast cells; after centrifugation at 5,000 g for 30 s, a supernatant was removed, the yeast cells were resuspended in 100 μL of physiological saline, spread evenly on DDO solid medium, and cultured for 3 d to 5 d. After single colonies grew, auxotrophic medium screening and verification was conducted, and the single colonies that had been successfully transferred into the corresponding plasmids were selected to TDO and QDO solid media containing x-α-gal, and the color change of the colony was observed (where blue represented interaction, white or red represented no interaction).

Referring to FIG. 5: in order to determine the interaction between GSS1 and the ABA hormone synthase aba1, structural and functional domains of the GSS1 were predicted online using NCBI, and GSS1 protein variants with 5 N-terminal and 3 C-terminal truncated were generated based on the prediction results. The 8 GSS1 protein variants were selected one by one through yeast two-hybrid. When 5 amino acids were deleted from the N-terminal or C-terminal of the sequence PIΔN-20aa, the GSS1 and aba1 no longer interacted, indicating that the shortest peptide segment that GSS1 interacted with aba1 was GSS1-PI-AN-20aa with a length of 28 amino acids. That is, the core sequence was: PDYKQKLRDSKAWESLETIGIPLDADVM (SEQ ID NO: 1). In FIG. 5, GSS1 could be divided into two segments, ALP and P, based on sequence prediction. The P was further divided into 3 segments from PI to PIII. 20 amino acids were further deleted from the N-terminal of PI to obtain the PIΔN-20aa. However, the PIΔN-20aa could not be further reduced from the N-terminal or the C-terminal.

### Example 2 Verification for the Influence of the Insect-Derived Short Peptide on Arabidopsis thaliana in the Present Disclosure

(1) Planting of Arabidopsis thaliana

In the present disclosure, Colombian wild type Arabidopsis thaliana was used, which was donated by the research team led by Professor You Minsheng of Fujian Agriculture and Forestry University, and preserved in the laboratory of Institute of Life Science, Gannan Normal University. The soil was mixed with peat soil: vermiculite in a ratio of 3:1. The seeds were spread on the soil surface after naturally absorbing water, or spotted on the surface of the ½ MS medium after sterilizing with 75% ethanol. Culture conditions were: temperature 24° C., relative humidity 60%, light conditions: 16 h light/8 h dark with light intensity of (6300±300) lx.

(2) Detection of Hormone Groups in Arabidopsis thaliana Treated with GSS1-PI Short Peptide

A synthetic short peptide of GSS1-PI with the corresponding concentration and mass was weighed and dissolved in 0.1% DMSO to obtain a 0.01 mg/mL treatment solution. A wild-type Arabidopsis thaliana plant of about four weeks old was taken, two cotyledons were collected and mechanically damaged to obtain three small holes, 2 μL of the GSS1-PI treatment solution was added dropwise into each small hole, while the control group was a solution of the same concentration prepared by synthesizing an unrelated short peptide GFP group (GFP-28aa: MSKGEELFTGVVPILVELDGDVNGHKFS, SEQ ID NO: 20) of the same length. After 24 h and 48 h of treatment, approximately 0.2 g of Arabidopsis thaliana leaves were collected, and each treatment had three biological replicates. The leaves were quickly placed in liquid nitrogen and stored in a −80° C. ultra-low temperature refrigerator for subsequent phytohormone group detection.

### (i) Sample Pre-Treatment

a. The cryopreserved biological samples were ground with a grinder (30 Hz, 1 min) until they were in a powder form.
b. 50 mg of the ground sample was weighed and added with 10 μL of an internal standard mixed solution with a concentration of 100 ng/ml and 1 mL of an extractant including methanol/water/formic acid (15:4:1, v/v/v), and mixed well.
c. After vortexing for 10 min, a resulting mixture was centrifuged at 4° C. and 12,000 r/min for 5 min, and a resulting supernatant was transferred to a new centrifuge tube for concentration.
d. After concentration, the concentrated solution was reconstituted with 100 μL of 80% methanol/water solution, the reconstituted solution was filtered through a 0.22 μm filter, and the filtrate was placed in a sampling bottle for LC-MS/MS analysis.

### (ii) Chromatography and Mass Spectrometry Data Acquisition Conditions

The data acquisition instrument system mainly included ultra-performance liquid chromatography (UPLC) (ExionLC™ AD, //sciex.com.cn/) and tandem mass spectrometry (MS/MS) (QTRAP® 6500+, //sciex.com.cn/).

Liquid phase conditions mainly included:
1) Chromatographic column: Waters ACQUITY UPLC HSS T3 C18 column (1.8 μm, 100 mm×2.1 mm i.d.);
2) Mobile phase: phase A, ultrapure water (added with 0.04% acetic acid); phase B, acetonitrile (added with 0.04% acetic acid);
3) Gradient elution program: 0 min A/B at 95:5 (V/V), 1.0 min A/B at 95:5 (V/V), 8.0 min at 5:95 (V/V), 9.0 min at 5:95 (V/V), 9.1 min at 95:5 (V/V), 12.0 min at 95:5 (V/V);
4) Flow rate: 0.35 mL/min; column temperature: 40° C.; injection volume: 2 μL.

Mass spectrometry conditions mainly included:

Electrospray ionization (ESI) ion source temperature was 550° C., the mass spectrometer voltage in the positive ion mode was 5,500 V, the mass spectrometer voltage in the negative ion mode was-4,500 V, and the curtain gas (CUR) was 35 psi. In the Q-Trap 6500+, each ion transition was scanned based on optimized declustering potential (DP) and collision energy (CE).

(iii) Qualitative and Quantitative Principles

A MWDB (Database) was constructed based on the standards, and the mass spectrometry detection data was qualitatively analyzed.

The quantificative analysis was accomplished using the multiple reaction monitoring mode (MRM, as shown below) of triple quadrupole mass spectrometry. In the MRM mode, the quadrupole first screened the precursor ions (parent ions) of the target substance and excluded ions corresponding to other molecular weight substances to initially eliminate interference; the precursor ions were induced to ionize in the collision chamber and then fragmented to form multiple fragment ions. The fragment ions were then filtered through triple quadrupole to select the required characteristic fragment ions. And the non-target ion interference was eliminated, so that the quantification was more accurate and reproducible. After obtaining the mass spectrometry data of different samples, the chromatographic peaks of all target substances were integrated and quantitatively analyzed through the standard curve.

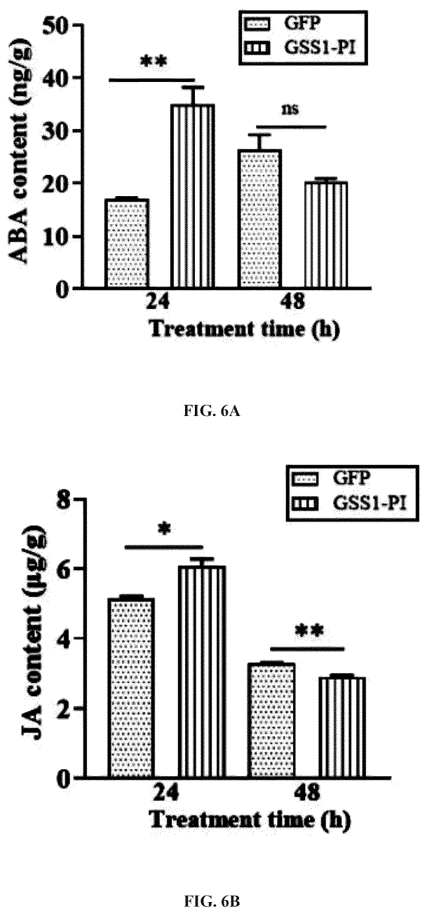

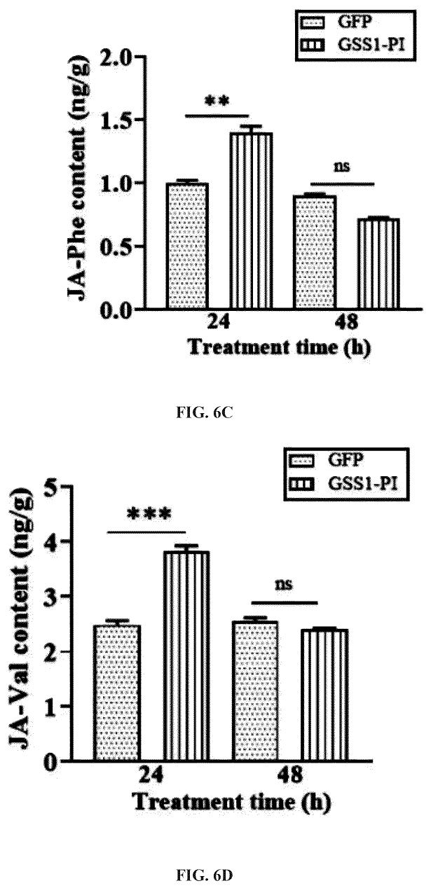

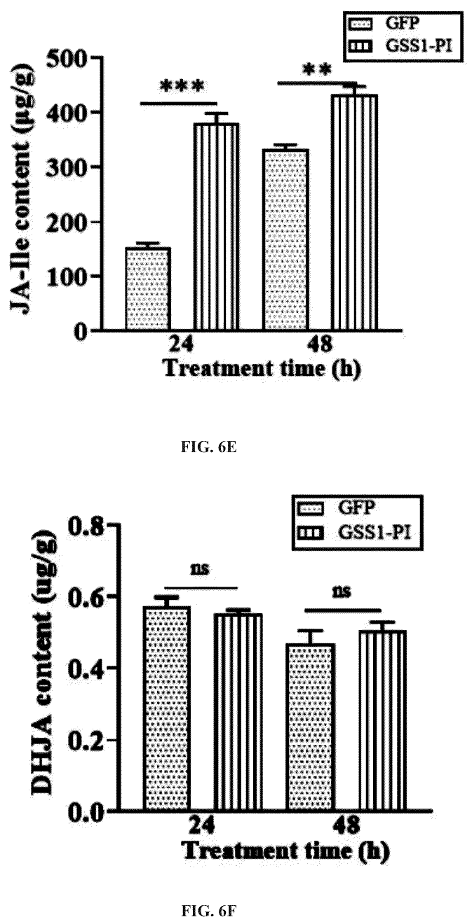

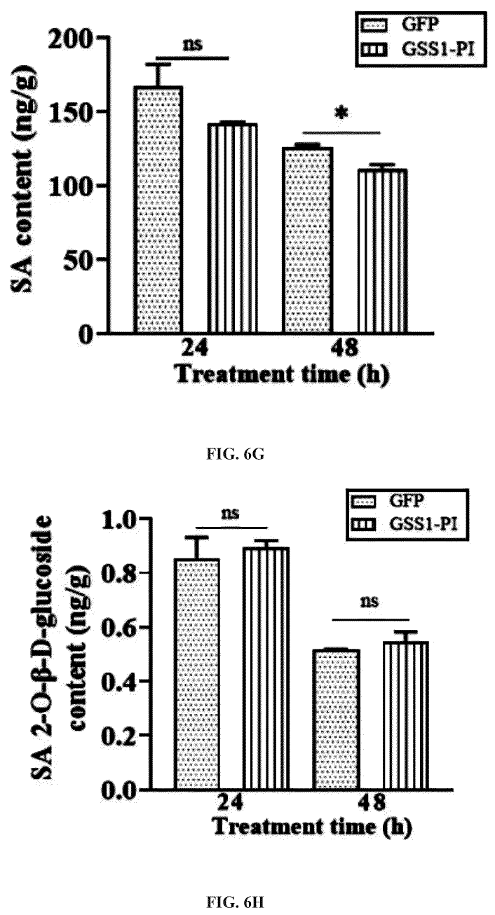

FIGS. 6A-6H showed a phytohormone content detected by GC-MS. Compared with the GFP control group, the ABA content of plants increased significantly after 24 h treatment with GSS-PI short peptide; however, after 48 h, there was an upward trend but no significant difference. There were two main modes of ABA-mediated insect resistance: direct mediation and indirect mediation. The direct mediation is reflected in the fact that ABA can directly positively or negatively regulate plant defense substances; while indirect mediation of plant insect resistance affects the plant's ability to resist insects by cooperating with other plant defense hormones JA/SA. This phenomenon is also called hormone cross-transmission. Therefore, the contents of JA and salicylic acid (SA) hormones in plants were also detected in the present disclosure. The JA content of plants increased significantly after 24 h of treatment, but showed a downward trend at 48 h; there was no significant change in SA content before and after treatment. This indicated that during the feeding of Plutella xylostella, the release of GSS1 protein could activate the plant's defense response, causing the plant ABA hormone to increase, and showing a positive regulatory trend with the plant defense hormone JA. However, SA did not participate in the GSS1-mediated insect resistance network, and its content showed no change.

### Example 3 Detection of Insect-Resistant Substance Content in Cruciferous Plants and its Insect-Resistant Effect

(1) Treatment of Arabidopsis thaliana with Synthetic Short Peptide GSS1-PI.

In this experiment, the materials used for metabonomics analysis and phytohormone detection are Arabidopsis thaliana samples with consistent growth status. Synthetic short peptide of GSS1-PI with the corresponding concentration and mass was weighed and dissolved in 0.1% DMSO to obtain a 0.01 mg/mL treatment solution. A wild-type Arabidopsis thaliana plant of about four weeks old was taken, two cotyledons were collected and mechanically damaged to obtain three small holes, 2 μL of the GSS1-PI solution was added dropwise into each small hole, while GFP solution was used as the control group. After 24 h and 48 h of treatment, approximately 0.2 g of Arabidopsis thaliana leaves were collected, and each treatment had three biological replicates. The leaves were quickly placed in liquid nitrogen and stored in a −80° C. ultra-low temperature refrigerator for subsequent phytohormone group detection and metabolomic analysis.

### (2) Detection of Metabolites

### 2.1 Metabolite Extraction

100 mg of Arabidopsis thaliana leaves was taken and added with liquid nitrogen for full grinding, and the fine powder was put in a 1.5 mL centrifuge tube. 500 μL of 80% methanol aqueous solution was pipette in the centrifuge tube and subjected to vortex oscillation to mix well, and the centrifuge tube was put on ice for 5 min. After standing, the mixture was centrifuged at 15000 g at 4° C. for 20 min. Then, 200 μL of supernatant was pipette and diluted with appropriate amount of mass spectrometry water until the methanol content in the solution was 53%. The dilution was centrifuged again for 20 min, the supernatant was retained and loaded for LC-MS analysis of the sample.

### 2.2 Instrument Parameters

### 2.3 Chromatographic Conditions

Related instruments were set as follows: chromatographic column temperature: 40° C.; injection flow rate: 0.2 mL/min; Positive ion mode: 0.1% formic acid for mobile phase A; mass grade methanol for mobile phase B; Negative ion mode: 5 mM ammonium acetate for mobile phase A, and mass grade methanol for mobile phase B.

### 2.4 Mass Spectrometry Conditions

The scanning range was m/z 100-1500: the setting of ESI signal source was as follows: spray voltage: 3.5 V; sheath gas flow rate: 35 psi; auxiliary gas flow rate: 10 L/min; ion transport tube temperature: 320° C.; iontophoresis RF frequency: 60; auxiliary heater temperature: 350° C.; MS/MS secondary scanning is set to dependency scanning data.

(3) Data Pretreatment and Metabolite Identification of Arabidopsis thaliana Treated with GSS-PI Short Peptide.

After the experiment was completed, the relevant data files were imported into the database search software for analysis and processing, and the residence time, mass-to-charge ratio and other related parameters of each metabolite were simply screened and checked. In order to identify more accurately, the different samples were peak-aligned, with the retention time deviation of 0.2 min and mass deviation of 5 ppm. Then peak extraction and peak area quantification were carried out to integrate the target ions, and then ion peaks were predicted by molecular formula and compared with local data. In order to remove the interference of background ions, the original quantitative results were standardized and blank sample controls were added. Finally, the relative quantitative results of the relevant metabolites were obtained and identified.

(4) Statistical Analysis of Metabolites of Arabidopsis thaliana Treated with GSS-PI Short Peptide.

Multivariate statistical analysis of metabolite data included two parts, component analysis (PCA) and partial least squares discriminant analysis (PLS-DA). The data was converted by metaX, a metabonomics processing software, to get the VIP value of each metabolite. In univariate analysis, the statistical significance (P value) between the two groups of metabolites was analyzed by t test, and the fold change of metabolites between the two groups, that is, FC value, was calculated. The default screening criteria for differential metabolites were VIP>1, P value<0.05 and FC≥2 or FC≤0.5.

The default volcano plot was draw using ggplot2 software, the metabolites of interest were screened based on two parameters, VIP value and −log 10^{(P value)} of the metabolites.

The cluster heat map was drawn by Pheatmap software, and the data between metabolite groups were normalized by Z-score. The correlation analysis of differential metabolites was carried out by using R language.

(5) Biological Experimental Observation after GSS-PI Short Peptide Treatment with Arabidopsis thaliana.
1) 60 newly molted third-instar larvae fed on artificial feed were divided into 3 groups, and 20 larvae in each group were fed on Arabidopsis thaliana plants treated with GSS-P1 short peptide or GFP control short peptide;
2) In order to prevent plants from being damaged mechanically or influenced by external factors, a simple interactive biological observation device for insect-plant interaction was used to eliminate potential interference factors;
3) After 3 days, the simple interactive biological observation device was opened, and the number of larvae (4th instar larvae) was observed and counted, the number of larvae surviving was recorded, and divided by the initial number of 20 larvae to obtain the daily survival rate of larvae.

A total of 27 differential metabolites were accumulated in Arabidopsis thaliana leaves after 24 h of GSS-PI short peptide treatment, including at most 7 kinds of lipid molecules and phenylpropanoids, 5 kinds of organic acids and derivatives thereof and 5 kinds of organic heterocyclic compounds, 2 kinds of organic oxygen compounds and 2 kinds of benzene ring compounds, and 1 kind of alkaloid and derivatives thereof. The top 5 compounds that up-regulated cumulative multiples were α-Linolenoyl ethanolamide, cytosine, madecassic acid, eriodictyol and methyl dihydrojasmonate. Among them, it had been shown that methyl dihydrojasmonate played a very important role in insect resistance (Table 5).

TABLE 5
Metabolites that differed significantly from the control group after 24 h of treatment.

| Compounds | SuperClass(HMDB) | log₂FC | Up/Down |
|---|---|---|---|
| α-Linolenoyl ethanolamide | Organic nitrogen compounds | 1.586 | up |
| Cytosine | Organoheterocyclic compounds | 1.096 | up |
| Madecassic acid | Lipids and lipid-like molecules | 1.041 | up |
| Eriodictyol | Phenylpropanoids and polyketides | 0.908 | up |
| Methyl dihydrojasmonate | Lipids and lipid-like molecules | 0.784 | up |
| (−)-chimonanthine | Organoheterocyclic compounds | 0.731 | up |
| Hesperidin | Phenylpropanoids and polyketides | 0.708 | up |
| Rhoifolin | Phenylpropanoids and polyketides | 0.699 | up |
| 2-Phenylacetamide | Benzenoids | 0.608 | up |
| Spectinomycin | Organoheterocyclic compounds | 0.579 | up |
| Quercetin 3-O-sophoroside | Organic acids and derivatives | −2.173 | down |
| Gracillin | Lipids and lipid-like molecules | −1.823 | down |
| Ptaquiloside | Organic oxygen compounds | −1.418 | down |
| 3,4,5-Trimethoxycinnamic acid | Phenylpropanoids and polyketides | −1.348 | down |
| Coumarin | Phenylpropanoids and polyketides | −1.039 | down |
| Tiglic acid | Lipids and lipid-like molecules | −0.663 | down |
| N-Methyltryptamine | Organoheterocyclic compounds | −0.651 | down |
| L-arginine | Organic acids and derivatives | −0.600 | down |
| cis-2-Decenoic acid | Lipids and lipid-like molecules | −0.599 | down |
| Hippuric acid | Benzenoids | −0.465 | down |

A total of 40 accumulated differential metabolites were identified after 48 h of GSS-PI short peptide treatment with Arabidopsis thaliana leaves, including 9 kinds of phenylpropanoids, 8 kinds of lipid molecules, 7 kinds of organic heterocyclic compounds, 6 kinds of benzenoids, 5 kinds of nucleosides and analogues thereof, 3 kinds of organic acids and derivatives thereof, and 2 kinds of organic oxygen compounds. The top 5 compounds that up-regulated cumulative multiples were cimifugin, neopterin, flavonoid (Camelliaside A), alcohol derivatives of pantothenic acid (DL-Panthenol), and benzamide (Table 6). Among them, flavonoid (Camelliaside A) played a significant role in insect resistance. Therefore, combined with the results of non-targeted metabolomics sequencing, it is believed that GSS1-P1 treatment can up-regulate the content of methyl dihydrojasmonate and flavonoid (Camelliaside A), thus mediating the insect resistance of cruciferous plants.

TABLE 6
Accumulation of differential metabolites between experimental
and control groups after 48 h of treatment

| Compounds | SuperClass(HMDB) | log₂FC | Up.Down |
|---|---|---|---|
| Cimifugin | Organoheterocyclic compounds | 1.872 | up |
| Neopterin | Organoheterocyclic compounds | 1.709 | up |
| Camelliaside A | Phenylpropanoids and polyketides | 1.640 | up |
| DL-Panthenol | Lipids and lipid-like molecules | 1.615 | up |
| Benzamide | Benzenoids | 1.564 | up |
| 2′-O-Methyladenosine | Nucleosides, nucleotides, and | 1.543 | up |
| | analogues | | |
| DL-Methionine | Organic acids and derivatives | 1.393 | up |
| Alpha-Mangostin | Organoheterocyclic compounds | 1.363 | up |
| 5′-Deoxy-5′- | Nucleosides, nucleotides, and | 1.330 | up |
| (Methylthio)Adenosine | analogues | | |
| Cucurbitacin E | Lipids and lipid-like molecules | 1.309 | up |
| 3,4,5-Trimethoxycinnamic acid | Phenylpropanoids and polyketides | −2.132 | down |
| Sclareolide | Organoheterocyclic compounds | −2.003 | down |
| Coumarin | Phenylpropanoids and polyketides | −1.930 | down |
| Allantoic acid | Organic acids and derivatives | −1.815 | down |
| Saikosaponin B4 | Lipids and lipid-like molecules | −1.655 | down |
| Scopoletin | Phenylpropanoids and polyketides | −1.518 | down |
| Sinapinic acid | Phenylpropanoids and polyketides | −1.203 | down |
| 5α-Dihydrotestosterone | Lipids and lipid-like molecules | −1.186 | down |
| glucuronide | | | |
| 20-Hydroxyecdysone | Lipids and lipid-like molecules | −1.153 | down |
| N-(p-Coumaroyl) serotonin | Organoheterocyclic compounds | −1.010 | down |

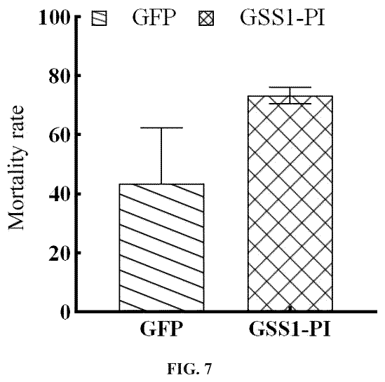

Referring to FIG. 7, Plutella xylostella larvae were fed with Arabidopsis thaliana treated with GSS1-PI short peptide for 3 days, the mortality rate of larvae was about 70%, which was significantly higher than that of the control group treated with GFP peptide (about 40%).

The above results verify that the insect-derived short peptide for improving an ABA content in a cruciferous plant in the present disclosure can improve the ABA hormone content in plants. Moreover, the increase in the hormone ABA content synergistically affects jasmonic acid (JA) hormone. The increase of plant endogenous hormones can further stimulate the content of downstream insect-resistant substances, and then lead to an increase in the mortality of Plutella xylostella. The technical scheme of the present disclosure provides important molecular evidence for the protein peptide to regulate plant hormones and further enhance plant stress resistance, indicating that this peptide segment shows a potential to enhance plant insect resistance in the future.

1. An insect-derived short peptide for improving an abscisic acid (ABA) content in a cruciferous plant, wherein the insect-derived short peptide for improving the ABA content in the cruciferous plant has an amino acid set forth in SEQ ID NO: 1.

2. A method for preparing the insect-derived short peptide for improving the ABA content in the cruciferous plant according to claim 1, wherein the method comprises the following steps: S1, constructing a yeast bait plasmid of a detoxifying enzyme glucosinolate sulfatase (GSS1) based on a yeast two-hybrid system; S2, screening the yeast bait plasmid of the GSS1 with an Arabidopsis thaliana-yeast library to obtain a plant protein zeaxanthin epoxidase aba1 that interacts with the GSS1; and S3, selecting a shortest interactive short peptide between the GSS1 and the aba1 based on a yeast one-to-one hybrid system.

3. A method for improving an abscisic acid (ABA) content in a cruciferous plant, comprising administering a product comprising the insect-derived short peptide of claim 1.

4. The method for improving an abscisic acid (ABA) content in a cruciferous plant of claim 3, wherein the insect-derived short peptide are dissolved in DMSO.

5. The method for improving an abscisic acid (ABA) content in a cruciferous plant of claim 3, wherein the insect-derived short peptide is prepared by the following steps: S1, constructing a yeast bait plasmid of a detoxifying enzyme glucosinolate sulfatase (GSS1) based on a yeast two-hybrid system; S2, screening the yeast bait plasmid of the GSS1 with an Arabidopsis thaliana-yeast library to obtain a plant protein zeaxanthin epoxidase aba1 that interacts with the GSS1; and S3, selecting a shortest interactive short peptide between the GSS1 and the aba1 based on a yeast one-to-one hybrid system.


SEQUENCE LISTING (20 sequences)

SEQ ID NO: 1 - protein, 28 aa, synthetic construct
  source 1..28: note = insect-derived short peptide
PDYKQKLRDSKAWESLETIGIPLDADVM

SEQ ID NO: 2 - DNA, 43 bp, synthetic construct
  source 1..43: mol_type = other DNA; note = Primer aba1-AD-F
ccatggaggccagtgaattcatgggttcaactccgttttgcta

SEQ ID NO: 3 - DNA, 42 bp, synthetic construct
  source 1..42: mol_type = other DNA; note = Primer aba1-AD-R
agctcgagctcgatggatccagctgtctgaagtaatttatcg

SEQ ID NO: 4 - DNA, 38 bp, synthetic construct
  source 1..38: mol_type = other DNA; note = Primer gss1-BD-F
tggccatggaggccgaattcatggccaccaagccccac

SEQ ID NO: 5 - DNA, 40 bp, synthetic construct
  source 1..40: mol_type = other DNA; note = Primer gss1-BD-R
cgctgcaggtcgacggatccttcggagtcggagtagggtt

SEQ ID NO: 6 - DNA, 38 bp, synthetic construct; identical to SEQ ID NO: 4
  source 1..38: mol_type = other DNA; note = Primer gss1-ALP-BD-F

SEQ ID NO: 7 - DNA, 40 bp, synthetic construct
  source 1..40: mol_type = other DNA; note = Primer gss1-ALP-BD-R
cgctgcaggtcgacggatccggttcccttgacgagcttgt

SEQ ID NO: 8 - DNA, 42 bp, synthetic construct
  source 1..42: mol_type = other DNA; note = Primer gss1-P-BD-F
tggccatggaggccgaattcatgatcgacgagtctctcagca

SEQ ID NO: 9 - DNA, 39 bp, synthetic construct
  source 1..39: mol_type = other DNA; note = Primer gss1-PI-BD-R
cgctgcaggtcgacggatcccatgacgtcagcgtccaga

SEQ ID NO: 10 - DNA, 38 bp, synthetic construct
  source 1..38: mol_type = other DNA; note = Primer gss1-PII-BD-F
tggccatggaggccgaattcatggctgaccgcgatgag

SEQ ID NO: 11 - DNA, 38 bp, synthetic construct
  source 1..38: mol_type = other DNA; note = Primer gss1-PII-BD-R
cgctgcaggtcgacggatccagccagctgaggaagctc

SEQ ID NO: 12 - DNA, 42 bp, synthetic construct
  source 1..42: mol_type = other DNA; note = Primer gss1-PIII-BD-F
tggccatggaggccgaattcatgcagatccttctgtaccgtc

SEQ ID NO: 13 - DNA, 40 bp, synthetic construct
  source 1..40: mol_type = other DNA; note = Primer gss1-PI-10aa-BD-F
tggccatggaggccgaattcatggaagacctccgtggcat

SEQ ID NO: 14 - DNA, 42 bp, synthetic construct
  source 1..42: mol_type = other DNA; note = Primer gss1-PI-20aa-BD-F
tggccatggaggccgaattcatgccagactacaagcagaagc

SEQ ID NO: 15 - DNA, 40 bp, synthetic construct
  source 1..40: mol_type = other DNA; note = Primer gss1-PII-10aa-BD-R
cgctgcaggtcgacggatccacgaagctcacaagggtcct

SEQ ID NO: 16 - DNA, 40 bp, synthetic construct
  source 1..40: mol_type = other DNA; note = Primer gss1-PI-25aa-BD-F
tggccatggaggccgaattcatgaagctgcgcgacagcaa

SEQ ID NO: 17 - DNA, 38 bp, synthetic construct
  source 1..38: mol_type = other DNA; note = Primer gss1-PI-20aa-BD5aa-R
cgctgcaggtcgacggatcccagagggatgccgatggt

SEQ ID NO: 18 - DNA, 21 bp, synthetic construct
  source 1..21: mol_type = other DNA; note = Primer pGBKT7-F
taatacgactcactatagggc

SEQ ID NO: 19 - DNA, 24 bp, synthetic construct
  source 1..24: mol_type = other DNA; note = Primer pGBKT7-R
ttttcgttttaaaacctaagagtc

SEQ ID NO: 20 - protein, 28 aa, synthetic construct
  source 1..28: note = unrelated short peptide GFP group : GFP-28aa
MSKGEELFTGVVPILVELDGDVNGHKFS